(19) United States
(12) Patent Application Publication
(10) Pub. No.: US 2026/0355033 A1
(43) Pub. Date: Nov. 12, 2026

(54) APPARATUS AND METHOD FOR USE WITH A PRESSURE VESSEL

(71) Applicant: DanMedical Limited, Oxfordshire (GB)

(72) Inventor: Ian Drysdale, Oxfordshire (GB)

(73) Assignee: DanMedical Limited, Oxfordshire (GB)

(21) Appl. No.: 19/670,739

(22) Filed: May 7, 2026

(30) Foreign Application Priority Data
May 8, 2025 (GB) 2507082.2

(51) Int. Cl. A61G 10/02; A61B 5/00; G16H 15/00

(52) U.S. Cl. CPC A61G 10/026; A61B 5/6888; G16H 15/00; A61B 2562/222; A61B 2562/227

(57) ABSTRACT

An apparatus for use within a pressure vessel for human occupancy (PVHO) such as a hyperbaric oxygen chamber. The apparatus comprises a communication interface connected, or connectable, to a computing device outside the PVHO, and a medical data module. The medical data module is configured to receive sensor signals from at least two sensors, the sensor signals being indicative of physiological measurements associated with a subject within the PVHO, generate medical data based on the sensor signals, and output the medical data to the computing device via the communication interface. Methods are also described.

### FIELD OF THE INVENTION

The invention relates to an apparatus and a method for outputting medical data from within a pressure vessel for human occupancy (PVHO).

### BACKGROUND OF THE INVENTION

In medical devices, such as patient monitors, physiological measurements may be performed, in which a cable is attached directly to a patent at one end by electrode wires to perform physiological tests, such as an electrocardiogram (ECG). A common design used to perform ECG measurements, consists of a number of electrode patches connected to the patient's skin in which voltage variations are recorded over a period of time, and the resulting signals are processed, stored and interpreted. The electrical signals sensed by the electrodes are commonly amplified and filtered in order to generate useful data. Although there are systems in the prior art for monitoring the physiological condition of a patient many of these prior art systems require a patient to wear a type of body monitor which then sends signals to a computer device such as a pda or a laptop.

Other forms of physiological measurements may also be performed, including measuring blood pressure and blood oxygen saturation, using corresponding sensors.

It may be desirable to take physiological measurements when a patient is within a pressure vessel for human occupancy (PVHO), such as a hyperbaric chamber. However, the sealed nature of PVHOs requires multiple sensor (and optionally power) cables to pass through the PVHO wall, which may be undesirable for structural, sealing, and commercial reasons. The challenges may be exacerbated if the PVHO is designed for producing an O₂-rich environment, where fire risks may be increased. Monoplace chambers (i.e., for only single subject) may further exacerbate challenges and risks due to limited space for sensors and associated equipment.

At least one object of the invention is to address one or more disadvantages associated with the prior art.

### SUMMARY OF THE INVENTION

In accordance with a first aspect, there is provided an apparatus for use within a pressure vessel for human occupancy, PVHO, the apparatus comprising:
a communication interface connected, or connectable, to a computing device outside the PVHO; and
a medical data module configured to:
   receive sensor signals from at least two sensors, the sensor signals being indicative of physiological measurements associated with a subject within the PVHO;
   generate medical data based on the sensor signals; and
   output the medical data to the computing device via the communication interface.

This arrangement may allow for the convenient collection of medical data from a subject within a PVHO and the transmission of the medical data to a computing device outside the PVHO for further processing.

The medical data module may comprise:
one or more medical data gathering modules configured to receive the sensor signals and generate the medical data; and
a medical data handling module operatively connected to receive the medical data from the medical data gathering modules, the medical data handling module being configured to buffer the transfer of the medical data between the medical data modules and the computing device via the communication interface.

Buffering the medical data may allow for improved and/or more flexible transfer of the medical data to the computing device via the communication interface.

The medical data handling module may comprise a microprocessor, a microprocessor and memory, a system programmable on a chip, or a field programmable gate array (FPGA).

The medical data handling module may comprise at least one first in first out (FIFO) memory buffer.

The medical data handling module may comprise a programmable read only memory, an electronically programmable read-only memory (EPROM), and/or an electronically erasable programmable read only memory (EEPROM), for delivering instructions to the medical data handling module.

The medical data handling module may be arranged to gather and store data in at least one predetermined group of data, such that the computing device may retrieve data from the medical data gathering module in one or more multiples of the at least one predetermined group of data.

The predetermined group of data may be N bits of data and/or T1 seconds worth of data.

The medical data handling module may be arranged to gather N bits of data every T1 seconds from the medical data gathering data and store the sampled bits of data in the predetermined group of data.

1/T1 may be the rate that the medical data handling module samples data from the at least one medical data gathering module.

One or more multiples of the predetermined groups of data may comprise one or more multiples of T1 seconds worth of data, and/or one or more multiples of N bits of data.

The communication interface may comprise a communication module configured to:
receive the medical data from the medical data module;
format the received medical data in accordance with a communication protocol; and
transmit the formatted medical data to the computing device.

Formatting the medical data in this manner may provide a simple, convenient and/or efficient way of transmitting, to the computing device, medical data based on sensor signals from multiple sensors. At least in some examples, and particularly where multiple sensors are employed, the use of a communication interface may reduce the number of conduits passing through the wall and/or door of the PVHO may be reduced, compared with (for example) passing a cable per sensor through the wall and/or door of the PVHO.

Formatting the sensor data may comprise packetizing the sensor data.

For example, when the sensor data takes the form of serial data values from a sensor, packetizing the sensor data may include forming packets, each packet comprising one or more of the serial data values and optionally one or more data values from a different sensor.

When the sensor data takes the form of a single value from a sensor, packetizing the sensor data may include forming packets, each packet comprising the single value and optionally one or more other data values from the same sensor and/or a different sensor.

The communication protocol may be a Universal Synchronous Bus, USB, communication protocol.

The communication interface may comprise a signal voltage conversion circuit configured to boost a voltage of the formatted medical data signal prior to its transmission to the computing device.

Optionally, the signal voltage conversion circuit may also be configured to reduce a voltage of signal data received at the communication interface from the computing device.

The communication interface may include at least one power supply connection connected, or connectable, to a power supply outside the PVHO, the power supply connection being configured to provide power from the power supply to at least one device, sensor, or other component within the PVHO.

The at least one power supply connection may include at least a first power supply connection configured to provide a power supply to the medical data module.

In examples, where the apparatus comprises a medical data gathering module and/or a medical data handling module, the power supply may be configured to power either or both of the medical data gathering module and/or a medical data handling module.

In examples, the power supply may be configured to power one more support circuits, interface circuits, or integrated circuits associated with the medical data module.

In examples, the power supply may be configured to power one or more of the sensors, and/or one or more other devices or components within the PVHO.

The at least one power supply connection may include at least a second power supply connection configured to provide a power supply to one or more of the sensors, and/or one or more other devices or components within the PVHO.

The at least one power supply connection may include at least:
a first power supply connection configured to supply power to at least a sub-component of the controller; and
a second power supply connection configured to supply power to one or more of the sensors, and/or one or more other devices or components within the PVHO,
wherein the first power supply connection is configured to supply power at a different voltage to that of the second power supply connection.

The communication interface may comprise a socket, the socket comprising one or more power terminals through which power from the at least one power supply passes, in use.

The socket may comprise one or more data terminals through which the medical data is transmitted to the computing device, in use.

The communication interface may comprise a socket, the socket comprising one or more data terminals through which the medical data is transmitted to the computing device, in use.

The socket may comprise one or more continuity terminals through which a continuity signal passes, in use, the continuity signal being useable by the computing device and/or the apparatus to determine whether the apparatus is connected to the computing device via the socket.

The socket may comprise a pair of the continuity terminals, the pair of continuity terminals being electrically connected to each other.

The communication interface may be a two-way communication interface, configured to receive signal data from the computing device.

The signal data may be configured to control any one or more of:
the communication interface;
the medical data module; and
the sensors.

The communication interface and the medical data module may be located within a unitary housing.

The apparatus may comprise a plurality of the medical data gathering modules, each of the medical data gathering modules being connected, or connectable, to a sensor.

The medical data gathering modules may comprise any one or more of an electrocardiogram data gathering module, a BP data gathering module, spirometry data gathering module, invasive or non-invasive blood pressure monitoring data gathering module; spirometry (lung function) data gathering module; pulse oximetry data gathering module; temperature data gathering module; both invasive and noninvasive blood pressure data gathering module; audiometry testing data gathering module; audio heart and/or lung sounds data gathering module; retinal testing data gathering module; ultrasound data gathering module; dermatology screening (image capture) data gathering module; imaging, tissue and/or wound care data gathering module; video endoscopy data gathering module; video for remote consultations data gathering module; video conference data gathering module; audio data gathering module; scaliometer (height) data gathering module; foetal heart Doppler ultrasound and/or audio acquisition and/or analysis data gathering module, an end tidal CO₂ (ETCO₂) gathering module, and a weighing scales data gathering module.

The medical data module may comprise at least one analogue-to-digital converter for digitising sensor data received from the sensors.

In accordance with a first aspect, there is provided a system comprising:
an apparatus according to the first aspect; and
one or more sensors connected, or connectable, to the medical data module.

The system may comprise a medical data cable comprising:
a first end connected, or connectable, to the communication interface; and
a second end distal to the first end, the second end being connected, or connectable, to a first PVHO socket within a chamber of the PVHO, the first PVHO socket being configured for operative connection to the computing device.

In accordance with a third aspect, there is provided a PVHO system comprising:
a PVHO; and
a system according to the second aspect.

In accordance with a fourth aspect, there is provided a PVHO system comprising:
a system according to the second aspect; and
a PVHO, the PVHO comprising the first PVHO socket.

The PVHO may comprise a second PVHO socket outside a housing of the PVHO, the second PVHO socket being operatively connected to the first PVHO socket.

In accordance with a fifth aspect, there is provided a method of outputting medical data from the interior of a PVHO chamber to a computing device located outside the PVHO chamber, the method comprising:
receiving, within the PVHO chamber, sensor signals from at least two sensors, the sensor signals being indicative of physiological measurements associated with a subject within the PVHO;
generating medical data based on the sensor signals; and outputting the medical data from inside the chamber to the computing device via a communication interface.

### BRIEF DESCRIPTION OF THE DRAWINGS

Aspects and implementations will now be described, without limitation and by way of example only, with reference to the accompanying drawings, in which:

FIG. 1 is a perspective view of a pressure vessel for human occupancy (PVHO) in the form of a hyperbaric oxygen chamber, according to an embodiment;

FIG. 2 is a schematic view of an apparatus, according to a further embodiment;

FIG. 3 is a schematic view of a system including the hyperbaric oxygen chamber of FIG. 1, the apparatus of FIG. 2, and a computing device, according to a further embodiment;

FIG. 4 is a schematic view of an example of the medical data module from the apparatus of FIG. 2;

FIG. 5 is a schematic view of the computing device shown in FIG. 3;

FIG. 6 is a schematic view of a further example of an apparatus, according to a further embodiment of the invention; and

FIG. 7 shows a method according to a further embodiment.

### DETAILED DESCRIPTION OF THE INVENTION

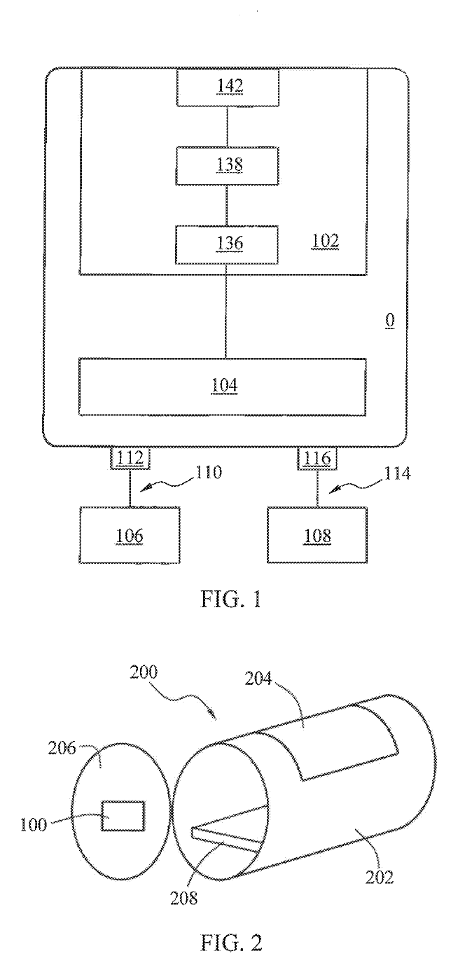

Referring to the drawings, FIG. 1 shows an apparatus 100. The apparatus 100 is designed for use within a pressure vessel for human occupancy (PVHO) 200, as shown in FIG. 2.

In the illustrated example, the PVHO 200 takes the form of a hyperbaric oxygen chamber designed to provide a pressurized, O₂-enriched environment for a human subject. The PVHO 200 includes a housing 202, including a transparent window 204 to allow a human occupant to see out and to allow technicians to see in. The PVHO 200 is closed by a door 206 (shown in an open state in FIG. 2). A bed 208 extends within the housing 202, allowing an occupant (not shown) to recline within the PVHO 200.

The apparatus 100 includes a communication interface 102. The communication interface 102 is connected, or connectable, to a computing device outside the PVHO 200, as described in more detail below. The connection to the computing device may be made directly, or via one or more intermediate circuits between the PVHO 200 and the computing device.

The apparatus 100 includes a medical data module 104. The medical data module 104 is configured to receive sensor signals from a first sensor 106 and a second sensor 108. The sensor signals are indicative of physiological measurements associated with a subject within the PVHO.

The first sensor 106 may, for example, be an electrocardiogram (ECG) sensor 106, which may include one or more ECG electrodes (not shown) that may be attached to the subject’s skin for measuring, for example, electrical activity. The ECG sensor 106 includes one or more electrode cables 110 terminating at a first plug 112, for connection to a corresponding socket of the apparatus 100, as described in more detail below.

The second sensor 108 may, for example, be a pulse oximetry (SpO₂) sensor 108, configured to be clipped over the end of the subject’s finger to measure blood oxygen saturation. The pulse oximetry sensor includes a cable 114 terminating at a second plug 116, for connection to a corresponding socket of the apparatus 100, described in more detail below.

The first sensor 106 and/or the second sensor 108 may take the form of any other sensors. The sensors may include any sensor configured to directly or indirectly sense or measure physiological parameters associated with the subject in the PVHO 200. For example, the sensors may include any one or more of an invasive or non-invasive blood pressure (IBP or NIBP) sensor, a spirometry (lung function) sensor, a pulse oximetry (SpO₂) sensor, a temperature sensor, an audiometry testing sensor, an audio heart and/or lung sound sensor, a retinal testing sensor, an ultrasound sensor, a dermatology screening sensor (such as an image capture device), an imaging, tissue and/or wound care sensor, a video endoscope, a video capture device, a video conference data gathering device, an audio data gathering device, a foetal heart Doppler ultrasound and/or audio acquisition and/or analysis data device, an end tidal CO₂ (ETCO₂) sensor, and a weighing scale.

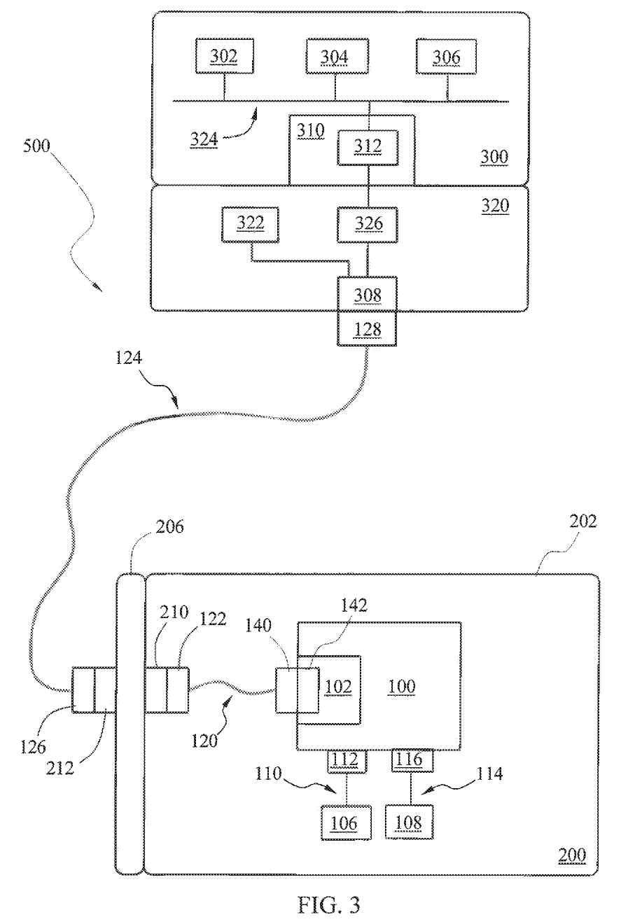

FIG. 3 shows a system 500 including the PVHO 200, the apparatus 100, and a computing device 300, and a housing 320.

The PVHO 200 includes a first PVHO socket 210 within the chamber of the PVHO 200. In the illustrated example, the first PVHO socket 210 is mounted to an inner surface of the door 206. In other examples, the first PVHO socket 210 may be mounted to an inner surface of a wall of the housing 202 or the window 204. The first PVHO socket 210 may be, for example, a 14-pin socket, although any socket having the required number of pins or contacts may be used in other examples.

A second PVHO socket 212 is disposed outside the chamber of the PVHO 200. In the illustrated example, the second PVHO socket 212 is mounted to an outer surface of the door 206. In other examples, the second PVHO socket 212 may be mounted to an outer surface of the PVHO housing 202 or the window 204. The second PVHO socket 212 may be, for example, a 14-pin socket, although any socket having the required number of pins or contacts may be used in other examples.

The first PVHO socket 210 is operatively connected to the second PVHO socket 212. For example, pins or contacts (not shown) within the first PVHO socket 210 may be electrically connected to corresponding pins or contacts (not shown) within the second PVHO socket 212.

To keep cable runs short, the first PVHO socket 210 and the second PVHO socket 212 may be positioned at the same location on the door 206, housing 202, or window 204, or at least nearby to each other. This may also the use of only a single aperture through the door 206, housing 204, or window 204, which may simplify sealing and reduce the chance of leaks.

In other examples, the first PVHO socket 210 and the second PVHO socket 212 may be positioned at different locations. For example, the first PVHO socket 210 may be mounted on an internal surface of the housing 202, and the second PVHO 212 may be mounted on an external surface of the door 206.

In the illustrated example, the computing device 300 takes the form of a desktop or laptop personal computer. The computing device 300 includes at least one core processor 302 operatively connected to a memory 304. The computing device 300 may include a non-volatile storage device such as a solid state drive (SSD) 306, for storing operating system data, program data, and medical data, processed medical data, and the like. The computing device 300 may be configured to run an operating system such as Microsoft Windows®, Apple Mac® or a Linux operating system.

The computing device 300 also includes a communication interface 310. The communication interface 310 includes a communication module in the form of a USB module 312. In the illustrated example, the USB module 312 is a USB master, which coordinates communication with the communication interface 102 of the apparatus 100.

The USB module 312 may be configured to operate in accordance with USB version 1.1, although any other appropriate USB version may be employed in other examples.

The processor 302, memory 304, SSD 306, and communication interface 310 communicate with each other by way of one or more internal buses 324.

In the illustrated example, there is provided a housing 320 that is separate from, but attached to form an integrated device with, the computing device 300. For example, the housing 320 may be configured to attach to the computing device 300.

The housing 320 includes a power control circuit 322, a USB control circuit 326, and a communication interface in the form of a socket 308, which includes pins or contacts (not shown) as described in more detail below. The socket 308 may be a 16-pin socket, although any socket having the required number of pins or contacts may be used in other examples.

Connections between the computing device 300 and components within the housing 320 may be made internally. That is, any connections (including cabling, plugs, sockets, terminations, and the like) may be made in a manner that renders them inaccessible without at least partly dismantling the computing device 300, the housing 320, or disconnecting them from each other. Such disassembly or disconnection may require the use of a tool, such as a screwdriver, which is intended to dissuade casual access to the components and connections within the housing 320. One example of an internal connection is the USB module 312. Instead of using an external USB socket to connect the USB power and signal lines of the USB module 312, an internal (i.e., within the computing device 300) USB connector, such as an onboard header or socket on the motherboard or the like, is used.

The computing device 300 may be configured to provide the normal functionality expected of a personal computer or laptop computer. In addition, the computing device 300 is configured to provide physiological measurement data processing capabilities. For example, the computing device 300 may be configured to do any of the following:
store the received medical data, for example on the SSD 306;
process the medical data, including filtering the medical data, and editing or cleaning the medical data (e.g., reducing noise, such as mains frequency noise); and
convert the medical data into visible form, such as one or more graphs, images, and animations, and present them to a technician.

Returning to FIG. 3, an internal cable 120 connects the communication interface 102 with the first PVHO socket 210. One end of the internal cable 120 terminates at a plug 122. The plug 122 includes pins or contacts (not shown) corresponding with pins or contacts (not shown) of the first PVHO socket 210. The plug 122 is shown plugged into the first PVHO socket 210. An opposite end of the internal cable 120 terminates at a plug 140. The plug 140 includes pins or contacts (not shown) corresponding with pins or contacts not shown within a socket 142 forming part of the communication interface 102. The socket 142 may be a 12-pin socket, although any socket having the required number of pins or contacts may be used in other examples.

An external cable 124 connects the computing device 300 to the second PVHO socket 212. One end of the external cable 124 terminates at a plug 126. The plug 126 includes pins or contacts (not shown) corresponding to pins or contacts (not shown) of the second PVHO socket 212. The plug 126 is shown plugged into the second PVHO socket 212. A second end of the external cable 124 terminates at a plug 128. The plug 128 includes pins or contacts (not shown) corresponding to pins or contacts (not shown) of the computing device socket 308. The plug 128 is shown plugged into the computing device socket 308.

The internal cable 120 and the external cable 124 operatively connect the apparatus 100 to the computing device 300 through the first PVHO socket 210 and the second PVHO socket 212.

Optionally, more than one conductor within the internal cable and/or the external cable may be electrically paralleled to improve current handling and reduce voltage drop. For example, two, three, four, or more conductors within either or both of the internal and external cables may be used to carry a power, signal, and/or ground current. This may be particularly useful for power-carrying conductors, where relatively high currents may result in greater voltage drop, especially over longer cable runs.

In use, a human subject (not shown) enters the PVHO 200 through the open door 206. The first sensor 106 and the second sensor 108 are attached to the subject in a known manner. The door 206 is closed. Oxygen is introduced to the PVHO 200 in a known manner. This may involve, for example, cycles of purging gas from within the housing 202 and pumping in oxygen to provide an O₂-rich, pressurized environment with the housing 202.

The first sensor 106 and the second sensor 108 generate sensor signals indicative of physiological measurements associated with the subject. The sensor signals are supplied to the medical data module 104. The medical data module generates medical data based on the received sensor signals and outputs the medical data to the computing device 300 via the communication interface 102.

The medical data module 104 may generate the medical data based on the sensor signals in any suitable manner. For example, FIG. 4 shows one example of the medical data module 104, in which the medical data module 104 includes a first medical data gathering module in the form of an ECG module 130, and a second medical data gathering module in the form of a pulse oximetry (SpO₂) module 132.

The ECG module 130 is configured and connected to receive sensor signals from the ECG sensor 106. The ECG module 130 includes operative components such as defibrillation protection, buffering and filtering circuitry, amplifier circuitry, a Wilson network, and analogue-to-digital conversion circuitry. Together, those operative components convert low-level analogue signals from the ECG sensor 106 into corresponding digital ECG data. The digital ECG data is a form of medical data.

The pulse oximetry module 132 is configured and connected to receive sensor signals from the pulse oximetry sensor 108. The pulse oximetry module 132 includes operative components such as a register for storing pulse oximetry values supplied by the pulse oximetry sensor 108. Alternatively, the sensor signals from the pulse oximetry sensor 108 may be received in analogue form, in which case the pulse oximetry module 132 may include operative components such as buffering and filtering circuitry, amplifier circuitry, analogue-to-digital conversion circuitry, and an output register or serial communication module. In any event, the pulse oximetry module 132 generates digital pulse oximetry data. The digital pulse oximetry data is a form of medical data.

The medical data module 104 of FIG. 4 also includes a medical data handling module 134. The medical data handling module 134 is operatively connected to receive the medical data (i.e., the digital ECG data and digital pulse oximetry data) from the medical data gathering modules (i.e., ECG module 130 and the pulse oximetry module 132).

The medical data handling module 134 is configured to buffer the transfer of the medical data between the medical data modules 130, 132 and the computing device 300 via the communication interface 102.

In the illustrated example, the medical data handling module 134 includes a field programmable gate array (FPGA). Optionally, the FPGA includes at least one first in first out (FIFO) memory buffer for buffering data received from the medical data modules 130, 132. The FPGA may be configured to sample data from external modules such as the medical data modules 130, 132, either directly or via one or more analogue-to-digital converters, as described in more detail below.

In other examples, the medical data handling module 134 may include one or more processors, one or more microprocessors, one or more memory units, one or more system-on-chip components, and/or any other processing component, and/or any combination thereof.

The medical data handling module 134 may also include one or more non-volatile memory units (not shown), such as a programmable read-only memory, an erasable programmable read-only memory (EPROM), or an electronically erasable programmable read only memory (EEPROM), for storing instructions to be implemented by the medical data handling module 134. Optionally, an EEPROM, such as a Serial Flash Memory (SFM), holds the FPGA program.

Optionally, the medical data handling module 134 may be arranged to gather and store data in at least one predetermined group of data. The computing device 300 is arranged to retrieve data from the medical data handling module 134 (via the communication interface) in one or more multiples of the at least one predetermined group of data. Optionally, the predetermined group of data is N bits of data and/or, optionally, T1 seconds worth of data. Optionally, the medical data handling module 134 is arranged to gather N bits of data every T1 seconds from the least one medical data gathering modules 130, 132 and store the sampled bits of data in the predetermined group of data. Optionally, 1/T1 is the rate that the medical data handling module samples data from the at least one medical data gathering module. Optionally, one or more multiples of the predetermined groups of data includes one or more multiples of T1 seconds worth of data, and/or one or more multiples of N bits of data. Optionally, N equals 24 to 54, 24, 48, 54, 56 or any multiple thereof. Optionally, gathered medical data is stored and retrieved by the computing device 300 intermittently from the at least one medical data handling module 134.

The communication interface 102 includes a communication module 136 configured to receive the medical data from the medical data module 104. Where the medical data module 104 includes the medical data handling module 134, the communication module 136 is configured to receive the medical data from the medical data handling module 134.

The communication module 136 is configured to format the received medical data in accordance with a communication protocol and transmit the formatted medical data to the computing device 300.

Where the medical data is received from the medical data module 104 in a non-packetized format, the communication module 136 may be configured to packetize the medical data. Where the medical data is received from the medical data module 104 in a packetized format, the communication module 136 may be configured to re-packetize the packetized medical data as part of formatting the medical data in accordance with the communication protocol.

The communication protocol may take any suitable form. In the illustrated example, the communication protocol is a universal synchronous bus, USB, communication protocol. The communication module 136 therefore includes a USB controller (not shown). However, for compactness and convenience, the various cables and plugs used to connect the apparatus 100 to the computing device 300 are not USB-standard plugs and cables. This allows a greater number of conductors to be used in, and types of data to be carried by, the cables than would be allowed under USB standards. This allows additional flexibility in terms of providing non-standard functionality, while still allowing the use of the well-established USB communications protocol for the packaging and transmitting the medical data.

Depending upon the implementation, the medical data may be packaged for multiplexing into USB protocol packets by the communication module 136 or the medical data handling module 134.

In view of the differential voltage signal transmission format provided under the USB standard, the various cables, plugs and sockets include a pair of signal conductors (not shown).

The communication interface 102 of FIG. 2 includes an optional signal voltage conversion circuit 138 configured to boost a voltage of the formatted medical data prior to its transmission to the computing device 300. For example, one USB standard uses +/-3.3 V differential signaling over a twisted pair. To ensure reliable transmission of the medical data through the internal cable 120, the external cable 124, the plugs 140, 122, 126, 128, the first PVHO socket 210 and the second PVHO socket 212, the signal voltage conversion circuit 138 boosts the +/-3.3 V signal to +/-5 V.

The signal voltage conversion circuit 138 may also be configured to reduce a voltage of signal data received at the communication interface 102 from the computing device 300. For example, the computing device 300 may be configured to send the signal data to the apparatus 100 via the communication interface 102. The signal data may be configured to control any one or more of the communication interface 102, the medical data module 104, and the sensors 106, 108.

Optionally, the communication interface 102 may be a two-way communication interface, configured to also receive the signal data from the computing device 300. Where a USB protocol is used, communications may be half-duplexed, such that the same twisted pair of conductors is used to transmit data in both directions, under the control of a master controller. In this regard, the master controller in the illustrated example is the USB module 312. In other examples, full-duplex communication may be enabled depending upon the communication protocol used.

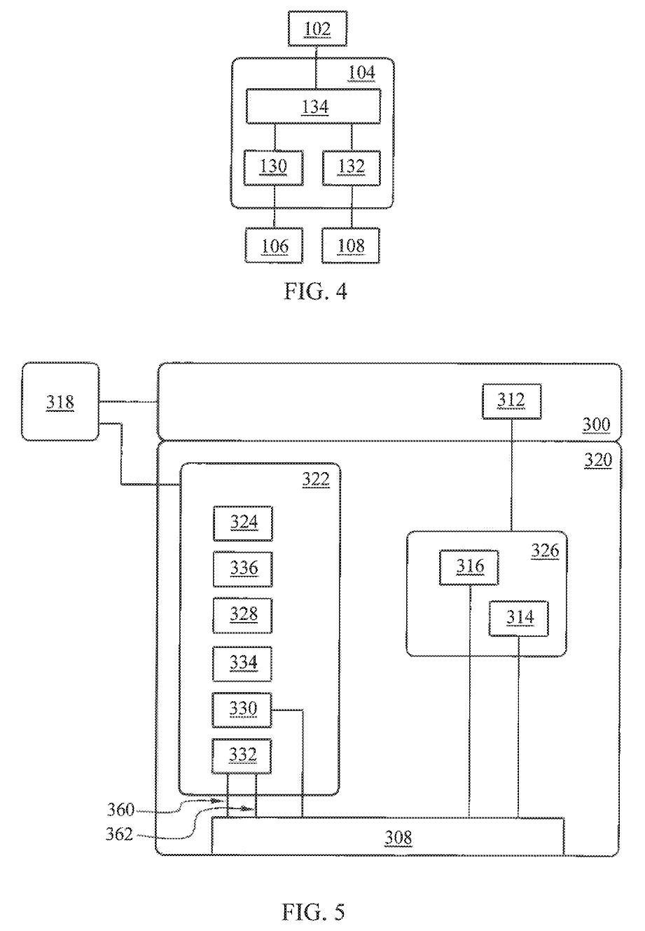

The USB control circuit 326 optionally includes one or more boost circuits. For example, as shown in FIG. 5, in the illustrated example, the USB control circuit 326 includes a signal converter circuit 314 configured to boost the USB signal voltage from +/-3.3 V to +/-5 V before transmission to the apparatus 100. This higher voltage provides for more reliable transmission of data through the internal cable 120, the external cable 124, the plugs 140, 122, 126, 128, the first PVHO socket 210, and the second PVHO socket 212.

The signal converter circuit 314 may also be configured to reduce the voltage of a data signal (e.g., the medical data) received from the apparatus 100. For example, if the communication interface 102 outputs differential data at +/-5 V, it may be necessary or desirable to reduce this voltage to +/-3.3 V for use by the USB module 312.

In the illustrated example, the USB control circuit 326 also includes a power boost circuit 316. Due to voltage losses across the internal cable 120, the external cable 124, the plugs 140, 122, 126, 128, the first PVHO socket 210, and the second PVHO socket 212, it may be desirable to boost the USB power supply voltage. For example, in the illustrated example, the USB module 312 outputs only 3.3 V from, for example, an internal USB pin on a motherboard or other circuit of the computing device 300. It may therefore be desirable to boost the voltage to 5 V with the power boost circuit 316.

In other examples, if a 5 V USB power supply is available, this may be used instead of the 3.3 V USB power supply. This may avoid the need for power boost circuit 316. However, some form of power boost may still be desirable to account for voltage losses over cabling, plugs, and sockets.

Optionally, the communication interface 102 of the apparatus 100 may include at least one power supply connection connected, or connectable, to a power supply outside the PVHO 200. Such a power supply connection is configured to provide power from the power supply to at least one device, sensor, or other component within the PVHO 200.

In the example of FIG. 3, the communication interface 102 is configured to receive a USB power signal in the form of a 5 V power supply. The USB power signal is supplied over conductor pairs (not shown) within the internal cable 120 and the external cable 124. The USB power signal is also passed through pins or contacts (not shown) within the plugs 140, 122, 126, 128, the first PVHO socket 210, and the second PVHO socket 212.

The 5 V power supply may be configured to power any combination of components within, or connected to, the apparatus 100. For example, the 5 V power supply may be configured to power the medical data module 104.

The 5 V power supply may be configured to power the first medical data gathering module 130, the second medical data gathering module 132, and/or the medical data handling module 134, where those modules are provided within the apparatus 100.

The 5 V power supply may be configured to power any one or more of the sensors 106, 108 connected to the medical data module 104.

The 5 V power supply may be configured to power the voltage conversion circuit 138.

In the example of FIG. 3, the computing device 300 may be a laptop 300. FIG. 5 shows an example in which the computing device 300 is a laptop 300. The laptop 300 is powered by a laptop power supply 318. The power supply 318 supplies DC power to the laptop 300 at a suitable voltage, such as 19 V. The power supply 318 may be powered by a mains power supply (not shown). Since the computing device 300 is configured to be connected to the apparatus 100, the power supply 318 may be a medical-grade power supply.

As well as powering the computing device 300, the output of the power supply 318 may be provided to a power control circuit 322. In the example of FIG. 5, the power control circuit 322 is disposed within the housing 320. This provides for a convenient and compact arrangement.

The power control circuit 322 includes one or more sub-circuits that use the 19 V power supply in various ways. For example, the sub-circuits may include any one or more of the following:
a switch 324 to isolate power from the other sub-circuits;
a thermal fuse 336 for cutting power in the event of an overcurrent and/or overheating event;
one or more voltage-sensing circuits 328 for cutting power in the event that the supply voltage is too high or too low;
a power limiting circuit 334 for limiting a maximum power supplied to the apparatus 100; and
a voltage convertor/regulator 330 for converting the supply voltage into a different voltage (e.g., converting the 19 V supply voltage to 12 V). An output of the voltage converter/regulator 330 may be connected to a contact or pin of the socket 308, enabling the provision of a 12 V power supply to the apparatus 100 through the cables, plugs, and sockets (i.e., the internal cable 120, the external cable 124, the plugs 140, 122, 126, 128, the first PVHO socket 210, and the second PVHO socket 212).

The sub-circuits may also include a connection detector 332 for determining whether all connections have been made between the apparatus 100 and the communication interface 310. In the example of FIGS. 3 and 5, the connection detector 332 outputs a continuity signal (e.g., a DC voltage) to one contact or pin within the socket 308, and monitors the voltage on another contact or pin within the socket 308, via continuity signal lines 360, 362. The socket 142 includes first and second continuity pins or contacts (not shown) electrically corresponding to respective pins or contacts with the socket 308 to which the connection detector 332 is connected. The continuity pins or contacts are electrically connected to each other.

When all the cables, plugs, and sockets (i.e., the internal cable 120, the external cable 124, the plugs 140, 122, 126, 128, the first PVHO socket 210, and the second PVHO socket 212) are correctly connected, a closed circuit is formed. The continuity signal (e.g., the DC voltage) from the connection detector 332 can pass through the continuity line 360, the corresponding conductors within the cables, plugs, and sockets (i.e., the internal cable 120, the external cable 124, the plugs 140, 122, 126, 128, the first PVHO socket 210, and the second PVHO socket 212) to the apparatus 100, cross the short at the continuity terminals, within the socket 142, return through corresponding conductors within the cables, plugs, and sockets (i.e., the internal cable 120, the external cable 124, the plugs 140, 122, 126, 128, the first PVHO socket 210, and the second PVHO socket 212), and pass through the continuity line 362, to the connection detector 332.

By detecting the signal on the return contact or pin within the socket 308, the connection detector 332 can confirm that all cables, plugs, and sockets are correctly connected. The connection detector 332 may then control a switch to connect power, such as the 12 V power from the voltage converter/regulator 330, to the relevant contact within the socket 308, thereby supplying that power to the apparatus 100.

If the continuity path is broken at any point, the circuit will not be closed, and the connection detector 332 will not enable the power supply.

The 12 V power supplied from the converter/regulator 330 may be configured to power any combination of components within, or connected to, the apparatus 100. For example, the 12 V supplied from the converter/regulator 330 may be configured to power the medical data module 104.

The 12 V power supplied from the converter/regulator 330 may be configured to power the first medical data gathering module 130, the second medical data gathering module 132, and/or the medical data handling module 134, where those modules are provided within the apparatus 100.

The 12 V power supplied from the converter/regulator 330 may be configured to power any one or more of the sensors 106, 108 connected to the medical data module 104.

The 12 V power supplied from the converter/regulator 330 may be supplied to the medical data module 104, which in turn distributes the power to one or more of the sensors, and/or other circuits and/or components within, or connected to, the apparatus 100.

The 12 V power supplied from the converter/regulator 330 power supply may be configured to power the signal voltage conversion circuit 138.

The 12 V power supplied from the converter regulator may also be stepped up or down by a voltage converter (not shown) within the apparatus 100, in order to provide components, such as sensors, with a power supply at a suitable voltage.

Optionally, ground for the 5 V USB power supply may be separated from the ground used for the 12 V power supply.

The communication interface 102 and the medical data module 104 may be located within a unitary housing. This provides a compact and convenient arrangement, which may be particularly useful within a PVHO that may already have limited space.

Although the examples described above use only two sensors, any number of such sensors may be connected to the apparatus in other examples. Similarly, any number of medical data gathering modules may be installed in other examples.

As well as being configured to buffer the sensor signals, generate the medical data based on the sensor signals, and transmit the medical data to the computing device 300, the apparatus 100 may also receive and process control signals from the computing device 300 (or any other connected device). For example, such control signals may control parameters associated with data acquisition (e.g., setting sampling rates, enabling/disabling sensors, starting/stopping acquisition, and/or starting/stopping data transmission). Such control signals may be transmitted over the same cables, plugs, and sockets (i.e., the internal cable 120, the external cable 124, the plugs 140, 122, 126, 128, the first PVHO socket 210, and the second PVHO socket 212) through which the medical data is transmitted. Alternatively, separate conductors may be provided for such control signals.

In alternative examples, one or more of the components within the housing 320 may be installed within the computing device instead. For example, any of such components may take the form of a card, circuit, or other device. The card, circuit or other device may plug into one or more existing interfaces, such as an IDE or PCI-E interface. Alternatively, or in the addition, the card, circuit, or other device may be soldered to a motherboard or other peripheral circuit board within the computer.

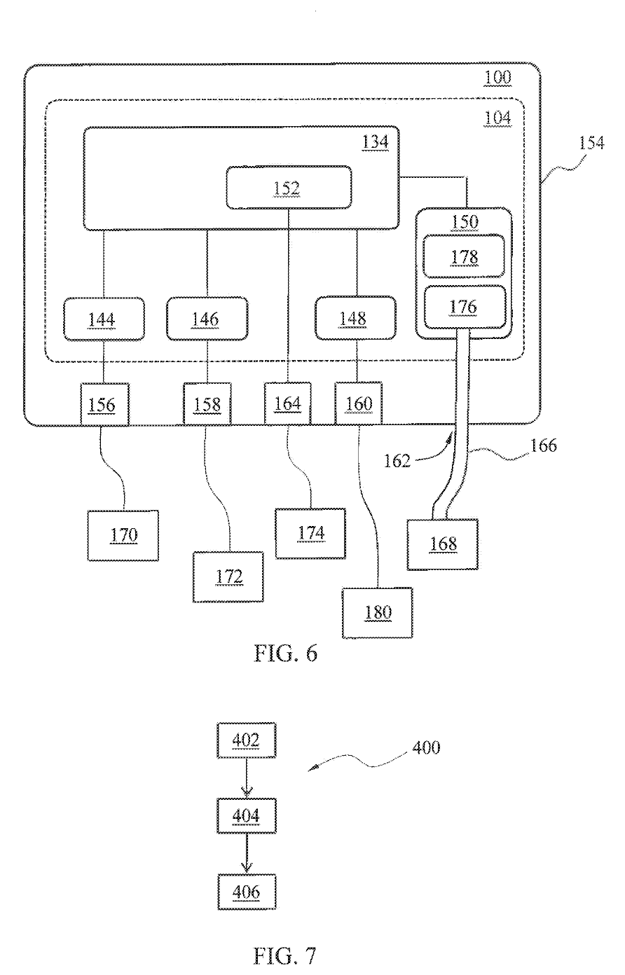

FIG. 6 shows a further example of an apparatus 100. For clarity, the communications interface 102 is omitted.

In the FIG. 6 example, the apparatus 100 includes a unitary housing 154, within which is disposed the medical data module 104. The medical data module 104 includes the medical data handling module 134.

The medical data module 104 includes:
a first medical data gathering module in the form of a temperature sensor module 144;
a second medical data gathering module in the form of an ECG module 146. The ECG module 146 includes the functionality described above in relation to the ECG module 130.
a third medical data gathering module in the form of an SpO₂ module 148. The SpO₂ module 148 includes the functionality described above in relation to the SpO₂ module 132;
a fourth medical data gathering module in the form of a non-invasive blood pressure (NIBP) module 150; and
a fifth medical data gathering module in the form of an end tidal CO₂ (ETCO₂) module 152. In contrast with the other medical data gathering modules, the end tidal CO₂ module 152 is embodied within the medical data handling module 134. That is, the FPGA within the medical data handling module 134 is configured to directly interface with an ETCO₂ sensor, rather than via a separate hardware data gathering module.

First, second, third, fourth, and fifth sockets are mounted to a wall of the housing 154. The first socket is a temperature socket 156, electrically connected to the temperature sensor module 144. The second socket is an ECG socket 158, electrically connected to the ECG module 146. The third socket is an SpO₂ socket 160, electrically connected to the SpO₂ module 148.

The fourth socket is an aperture 162 extending through the wall of the housing 154. A pneumatic NIBP hose 166 extends from the NIPB module 150 through the aperture 162, terminating at an NIBP cuff 168. A grommet (not shown) may surround and seal the NIBP hose 166 where it passes through the aperture 162. The NIBP module 150 includes a pump 176 driven by a brushless motor 178. The pump 176 provides the pressure required to inflate the NIBP cuff 168. Using a brushless motor reduces fire risks in an oxygen-rich environment, due to the sparkless nature of brushless switching

The fifth socket is an ETCO₂ socket 164, electrically connected to the medical data handling module 134.

A temperature sensor 170 is connected to the temperature socket 156, ECG electrodes 172 are connected to the ECG socket 158, an ETCO₂ sensor 174 is connected to the ETCO₂ socket 164, and an SpO₂ sensor 180 is connected to the SpO₂ socket 160.

In use, the various sensors take physiological measurements of a subject within the PVHO 200. The measurements may be taken continuously, periodically, or under the control of a technician or a program running on the apparatus 100 or the computing device 300. For example, the NIBP cuff 168 may periodically be inflated and a blood pressure reading taken in a known manner.

The measurements taken by the various sensors are supplied to the medical data handling module 134 by way of the various sockets and sensor modules. The medical data handling module 134 outputs the medical data to the communications interface (not shown) for transmission to the computing device 300, for example as described above.

FIG. 7 shows a method 400 of outputting medical data from the interior of a PVHO chamber (such as PVHO chamber 200) to a computing device (such as computing device 300) located outside the PVHO chamber. The method 400 includes:
receiving 402, within the PVHO chamber, sensor signals from at least two sensors, the sensor signals being indicative of physiological measurements associated with a subject within the PVHO;
generating 404 medical data based on the sensor signals; and
outputting 406 the medical data from inside the chamber to the computing device via a communication interface.

Variants of the method 400 will be understood from the functional description of the apparatus 100 and other hardware of FIGS. 1-6.

In the illustrated example, the PVHO 200 takes the form of a hyperbaric chamber designed to provide a pressurized, O₂-rich environment for a human subject. In other examples, different forms of PVHO may be used. For example, higher or lower pressure PVHOs may be employed. Different gas combinations may also be employed, depending upon the purpose of the PVHO.

In addition to physiological sensors, the apparatus 100 may be configured to receive sensor signals from one or more other sensors configured to sense parameters that are not related, or only indirectly related, to physiology. For example, air temperature within the PVHO may affect physiological parameters such as core temperature, sweating, and the subject’s perceived comfort. Accordingly, it may be useful to sense the air temperature with a thermometer and provide air temperature data.

Circuit boards within the apparatus 100 may be conformal coated, to reduce fire risks.

Although various sockets and plugs are described, in other examples, one or more of the socket/plug pairs may be replaced with a hard-wired connection or another connection type.

Although electrical conductors are described, in other examples, different data transfer mechanisms may be employed for at least some of the data path between the apparatus 100 and the computing device 300. For example, optic fiber communication may be used for at least some of the path.

All modules and circuits described herein may take the form of be hardware, software, firmware, or any suitable combination thereof. For example, in examples that describe medical data gathering modules and a medical data handling module, it will be understood that one or more components of one or more of the medical data gathering modules may be implemented as part of the same hardware that implements some or all of the components of the medical data handling module, and vice versa.

Any other form of suitable computing device may be used, including tablet computing devices, mobile telecommunications devices such as smartphones, and special purpose computing devices configured specifically to operate as described herein.

Although the invention has been described with reference to a number of specific implementations, the skilled person will appreciate that the invention may be embodied in many other forms.

1. An apparatus for use within a pressure vessel for human occupancy, PVHO, the apparatus comprising:
a communication interface connected, or connectable, to a computing device outside the PVHO; and
a medical data module configured to:
receive sensor signals from at least two sensors, the sensor signals being indicative of physiological measurements associated with a subject within the PVHO;
generate medical data based on the sensor signals; and
output the medical data to the computing device via the communication interface.

2. The apparatus according to claim 1, wherein the medical data module comprises:
one or more medical data gathering modules configured to receive the sensor signals and generate the medical data; and
a medical data handling module operatively connected to receive the medical data from the medical data gathering modules, the medical data handling module being configured to buffer the transfer of the medical data between the medical data modules and the computing device via the communication interface.

3. The apparatus according to claim 1, wherein the communication interface comprises a communication module configured to:
receive the medical data from the medical data module;
format the received medical data in accordance with a communication protocol; and
transmit the formatted medical data to the computing device.

4. The apparatus according to claim 3, wherein:
formatting the sensor data comprises packetizing the sensor data.

5. The apparatus according to claim 1, wherein:
the communication interface comprises a signal voltage conversion circuit configured to boost a voltage of the formatted medical data signal prior to its transmission to the computing device.

6. The apparatus according to claim 1, wherein:
the communication interface includes at least one power supply connection connected, or connectable, to a power supply outside the PVHO,
the power supply connection being configured to provide power from the power supply to at least one device, sensor, or other component within the PVHO.

7. The apparatus according to claim 6, wherein the at least one power supply connection includes at least a first power supply connection configured to provide a power supply to the medical data module.

8. The apparatus according to claim 6, wherein the at least one power supply connection includes at least a second power supply connection configured to provide a power supply to one or more of the sensors, and/or one or more other devices or components within the PVHO.

9. The apparatus according to claim 6, wherein the at least one power supply connection includes at least:
a first power supply connection configured to supply power to at least a sub-component of the controller; and
a second power supply connection configured to supply power to one or more of the sensors, and/or one or more other devices or components within the PVHO,
wherein the first power supply connection is configured to supply power at a different voltage to that of the second power supply connection.

10. The apparatus according to claim 6, wherein:
the communication interface comprises a socket, the socket comprising one or more power terminals through which power from the at least one power supply passes, in use.

11. The apparatus according to claim 10, wherein:
the socket comprises one or more data terminals through which the medical data is transmitted to the computing device, in use.

12. The apparatus according to claim 1, wherein:
the communication interface comprises a socket, the socket comprising one or more data terminals through which the medical data is transmitted to the computing device, in use.

13. The apparatus according to claim 11, wherein:
the socket comprises one or more continuity terminals through which a continuity signal passes, in use, the continuity signal being useable by the computing device and/or the apparatus to determine whether the apparatus is connected to the computing device via the socket.

14. The apparatus according to claim 13, wherein:
the socket comprises a pair of the continuity terminals, the pair of continuity terminals being electrically connected to each other.

15. The apparatus according to claim 1, wherein:
the communication interface is a two-way communication interface, configured to receive signal data from the computing device.

16. The apparatus according to claim 15, wherein:
the signal data is configured to control any one or more of:
the communication interface;
the medical data module; and
the sensors.

17. The apparatus according to claim 1, comprising a plurality of the medical data gathering modules, each of the medical data gathering modules being connected, or connectable, to a sensor.

18. The apparatus according to claim 17, wherein the medical data gathering modules comprise any one or more of an electrocardiogram data gathering module, a BP data gathering module, spirometry data gathering module, invasive or non-invasive blood pressure monitoring data gathering module; spirometry (lung function) data gathering module; pulse oximetry data gathering module; temperature data gathering module; both invasive and noninvasive blood pressure data gathering module; audiometry testing data gathering module; audio heart and/or lung sounds data gathering module; retinal testing data gathering module; ultrasound data gathering module; dermatology screening (image capture) data gathering module; imaging, tissue and/or wound care data gathering module; video endoscopy data gathering module; video for remote consultations data gathering module; video conference data gathering module; audio data gathering module; scaliometer (height) data gathering module; foetal heart Doppler ultrasound and/or audio acquisition and/or analysis data gathering module, an end tidal CO₂ (ETCO₂) gathering module, and a weighing scales data gathering module.

19. The apparatus according to claim 1, wherein the medical data module comprises at least one analogue-to-digital converter for digitising sensor data received from the sensors.

20. A system comprising:
the apparatus of claim 1; and
one or more sensors connected, or connectable, to the medical data module.

21. The system according to claim 20, comprising a medical data cable comprising:
a first end connected, or connectable, to the communication interface; and
a second end distal to the first end, the second end being connected, or connectable, to a first PVHO socket within a chamber of the PVHO, the first PVHO socket being configured for operative connection to the computing device.

22. A method of outputting medical data from the interior of a PVHO chamber to a computing device located outside the PVHO chamber, the method comprising:
receiving, within the PVHO chamber, sensor signals from at least two sensors, the sensor signals being indicative of physiological measurements associated with a subject within the PVHO;
generating medical data based on the sensor signals; and
outputting the medical data from inside the chamber to the computing device via a communication interface.
